(19) United States
(12) Patent Application Publication
(10) Pub. No.: US 2019/0090807 A1
(43) Pub. Date: Mar. 28, 2019

(54) COMPOSITIONS AND METHODS FOR DETECTING HEAD TRAUMA

(71) Applicant: RONALD LANE GOODE, II, WATERTOWN, MA (US)

(72) Inventor: RONALD LANE GOODE, II, WATERTOWN, MA (US)

(21) Appl. No.: 15/714,908

(22) Filed: Sep. 25, 2017

(51) Int. Cl. A61B 5/00; A61B 5/103

(52) U.S. Cl. CPC A61B 5/6802; A61B 5/682; A61B 2503/10; A61B 5/103

(57) ABSTRACT

The invention provides a mouth guard comprising an impact indicator that changes color when a blow to the head occurs that exceeds a predetermined level or that is of sufficient force to induce a concussion.

### BACKGROUND OF THE INVENTION

News reports and studies reveal an ever-increasing number of athletes, at all levels of play, are experiencing concussions and concussion related issues (such as: memory and cognitive issues, dementia, Alzheimer's, depression and chronic traumatic encephalopathy “CTE”). The effects of multiple concussions may also have long last detrimental effects on memory and academic performance in non-professional athletes. Nearly 300,000 sport-related traumatic brain injuries occur annually in the United States among people aged 15-24 years of age. There may be an even larger number of brain injuries that go unrecognized because the symptoms of concussion vary and may take hours or days to appear. Moreover, coaches of youth sports teams may not have the training or experience to recognize when a player has suffered a concussion. Though high-cost devices are on the market to assist in the recognition of severe blows to the head they are priced beyond the means of most individuals. A need exists for a straightforward and inexpensive way of identifying athletes who have suffered a blow to the head that is of sufficient force to induce a concussion.

### SUMMARY OF THE INVENTION

As described below, the present invention features a mouth guard comprising an impact indicator that changes color when the wearer of the device sustains an impact that exceeds a predetermined level or that is of sufficient force to induce a concussion. Advantageously, the mouth guard of the invention is inexpensive and, therefore, accessible to a wide variety of non-professional athletes.

In one aspect, the invention provides a mouth guard containing a U-shaped structure having an inner wall and an outer wall, wherein the inner and outer walls are connected to each other by a base forming a channel between said inner and outer walls, which are designed for the upper teeth of a user, wherein the mouth guard contains an impact indicator. In one embodiment, the outer wall of the U-shaped structure contains one or more impact detectors. In another embodiment, a shield extends from the center of the outer wall of the U-shaped structure, and the shield contains one or more impact detectors. In another embodiment, a strap extends from the center of the shield and the strap contains one or more impact detectors. In another embodiment, the U-shaped structure contains a conduit extending between the inner and outer walls to facilitate breathing or drinking through the shield. In another embodiment, the impact detector is embedded in or adheres to the outer wall or the inner wall of the U-shaped structure. In another embodiment, the impact detector provides a visual signal or flavor signal indicating that an impact of a predetermined level has occurred. In another embodiment, the impact detector provides a visual signal or flavor signal indicating that an impact of between 20 Gs and 105 Gs has occurred. In another embodiment, the impact detector provides a visual signal at a force selected from the group consisting of 20 Gs, 50 Gs, 60 Gs, 80 Gs, 90 Gs, and 100 Gs. In another embodiment, the mouth guard contains 2 or more impact detectors each of which provides a different visual signal or flavor signal at a predetermined level of force. In another embodiment, one impact detector provides a visual signal at a force under 60 Gs and the second impact detector provides a visual signal at a force greater than 60 Gs. In another embodiment, the impact detector contains a flavor packet that ruptures upon impact at a predetermined level. In another embodiment, the flavor packet ruptures at a force selected from the group consisting of 20 Gs, 50 Gs, 60 Gs, 80 Gs, 90 Gs, and 100 Gs. In another embodiment, the impact detector contains liquid surface tension technology.

In another aspect, the invention provides a method for detecting a force of a predetermined level applied to a subject, the method involving contacting a subject wearing the mouth guard of a previous aspect with a force, and detecting the presence or absence of a visual signal in the impact detector, thereby indicating that the force applied was at or was not at the predetermined level. In one embodiment, the presence of a visual sign indicates the force applied to the subject was sufficient to cause a concussion and the absence of a visual signal indicates the force applied to the subject was not sufficient to cause a concussion.

In another aspect, the invention provides a method for detecting a concussion in a subject, the method involving contacting a subject wearing the mouth guard of claim 1 with a force, and detecting the presence or absence of a visual signal in the impact detector, wherein the presence of a color change indicates that the subject has sustained a concussion and the absence of a color change indicates that the subject has not sustained a concussion.

In another aspect, the invention provides a method for a subject to detect a force of a predetermined level applied to the subject, the method involving contacting a subject wearing the mouth guard of claim 1 with a force, and detecting the presence or absence of a flavor signal, wherein the presence of a flavor signal indicates that the force applied was at the predetermined level and the absence of a flavor signal indicates that the force applied was below the predetermined level.

Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence or absence of force sufficient to cause concussion.

By “reference” is meant a standard or control condition.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the term “rupture” refers to breakage of the membrane in response to impact. In one embodiment, the rupture is by bursting, splitting, cracking, rending, severing, fracturing, tearing, cleaving, forcing open, puncturing, splitting, or ripping. The rupture occurs only to the extent that flavor is released after rupture of the membrane.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a photograph showing an exemplary mouth guard comprising an impact indicator.

FIG. 2 is a photograph showing an exemplary mouth guard configured as a pacifier.

### DETAILED DESCRIPTION OF THE INVENTION

The invention provides a mouth guard comprising one or more impact indicators that changes color when an impact occurs that exceeds a predetermined level or that is of sufficient force to induce a concussion.

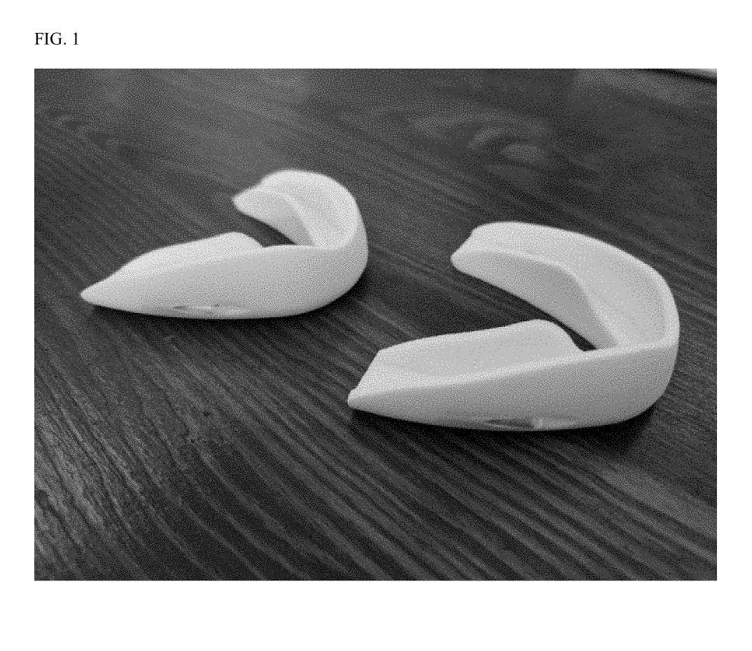

FIG. 1 illustrates an embodiment of a mouth guard comprising an impact detector.

### Concussion

A concussion may occur in response to a direct bump, blow or jolt to the head, face, neck or elsewhere on the body, internal movement of the brain caused by a direct or rotational force (such as whiplash), or multiple sustained smaller impacts to the brain. A concussion occurs when there is an alteration in conscious awareness after head trauma. Concussion can result from blows of varying force. In general, most concussions occur when the head receives an impact of approximately 90-100 Gs, but can occur with much less g-force. In one embodiment, repeated low impact blows (e.g., 20 Gs, 30 Gs, 40 Gs, 50 Gs, 60 Gs, 70 Gs, 80 Gs, 90 Gs, or 100 Gs) can cause a concussion. For example, repeated blows to the head of just 20 Gs, which is the force associated with the impact of a soccer ball, may cause a concussion.

### Mouth Guard

The size and/or dimensions of the mouth guard allow the mouth guard to accommodate a variety of different individuals having different jaw sizes or jaw or tooth configurations. The mouth guard can be configured to protect the upper teeth, lower teeth or both.

The mouth guards of the present invention may be molded out of materials such as rubber, plastic, polymers, as well as other man-made materials. The materials used may be varied to the desired resiliency or rigidity. In particular embodiments, the mouth guard is formed from a material selected from the group consisting of ethyl vinyl acetate (EVA), polyurethane, thermoplastic polyurethane, polystyrene, polypropylene, high-density polyethylene, polycaprolactone and rubber. Polypropylene if used exhibits a rigid character in that it holds its shape.

The mouth guard can also be made, in part, from thermal activated materials that soften when heated so as to receive an imprint of the teeth of a user and retain the imprint when cooled. This would aid in making a custom fit mouth guard for a user by placing the mouth guard in warm water so as to soften the thermal activated material, pressing against the user's teeth and holding it in place until the mold is cooled and the imprint is retained. In one embodiment, the impact detector is embedded in the thermal activated material prior to heating.

In one embodiment, the mouth guard is configured in the form of a pacifier as illustrated, for example, in D732,675, which is incorporated herein by reference.

### Impact Detector

The mouth guard comprises an impact detector that detects an impact at a pre-determined level and undergoes a color change when an impact of sufficient force is applied. Various types of impact detectors may be employed in this invention. One suitable mechanical impact detector employs liquid surface tension technology as disclosed in U.S. Pat. Nos. 4,068,613; 4,125,085; 4,177,751; 4,219,708; and 4,239,014, each of which is incorporated herein by reference. The mechanical shock detector is of the type disclosed in these patents and is sold by Detectors, Inc., of Graham, Tex. under the trademark Shockwatch™.

As depicted in FIG. 1, a suitable mechanical shock detector includes a hollow tube having a bore extending along the tube's longitudinal axis. At least two materials are disposed in the bore. The ends of the bore are closed by seals. There is a transparent or translucent light permeable section in at least one end of the tube. This tube has a relatively small internal diameter such that the bore may readily hold a droplet of liquid. If the entire tube is transparent, an opaque band may be employed to conceal the material until an interfacial boundary or barrier between the materials and has been ruptured by an impact exceeding, for example, 100 g's. The two materials may be, respectively, a gas and a liquid and are chosen such that the droplet of the liquid forming the material is held near the center of the tube by discrete slugs of gas filling the ends of the tube.

The mouth guard comprising the impact detector provides for the detection of impacts surpassing a predetermined level. Following an impact, the mouth guard is inspected to determine whether a color change has occurred. If no color change has occurred, the player may return to play. If a color change has returned, the player may not be returned to play, but will be referred for medical evaluation.

In one embodiment, an impact detector comprises a flavorant that ruptures upon impact surpassing a predetermined level. This allows a subject to identify when the subject has sustained an impact greater than the predetermined level.

If desired, the mouth guard is configured with multiple (e.g., 1, 2, 3, 4, 5) impact detectors, wherein each impact detector is set to undergo a color change at different predetermined level. In one embodiment, the predetermined level is 0.5, 1, 5, 10, 15, 20, 25, 50 or 75, 90, 95, 100, 110, 125, or 150 G.

The impact detector is fixed to the mouth guard using any means known in the art. In one embodiment, the impact detector is mounted on the mouth guard, adheres to the mouth guard, or is embedded within the mouth guard.

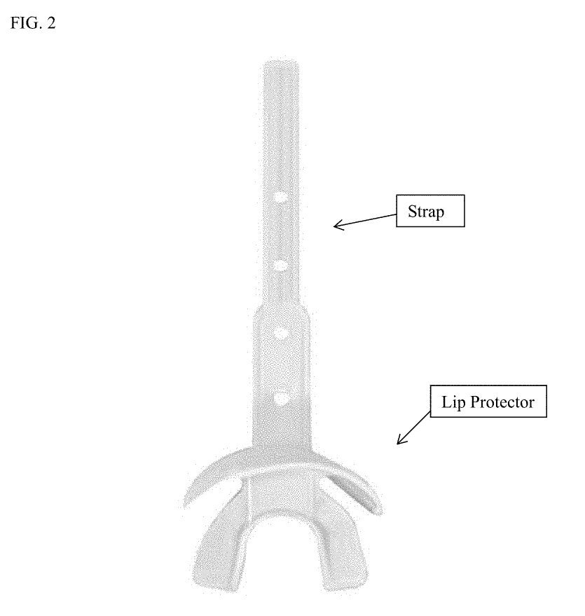

The impact detector may be mounted on virtually any surface of the mouth guard. In one embodiment, where the impact detector is a U-shaped structure, the impact detector is fixed to the outer wall or is fixed to the inner wall.

Where the mouth guard is configured as a pacifier, the impact detector is fixed on the portion of the mouth guard that protects the lips, is fixed on an outer wall of the U-shaped structure that protects the teeth, or is fixed on a strap that connects the mouth guard to the helmet (FIG. 2). Advantageously, when positioned on the lip protector or the strap, the impact detector is readily visible to third parties, such as parents, coaches, trainers, and medical personnel or is visible to the subject wearing the mouth guard when viewed in a mirror.

In another embodiment, the mouth guard is configured as shown in U.S. Pat. No. 8,931,488, which is hereby incorporated by reference in its entirety, to include a conduit extending between the inner and outer walls designed to facilitate breathing or drinking through the passage. In this configuration, the impact detector is fixed on the portion of the mouth guard that protects the lips, is fixed on an outer wall of the U-shaped structure that protects the teeth, or is fixed on an outer portion of the conduit.

### Flavor Packet

In one embodiment, the mouth guard comprises an impact detector in the form of a packet comprising, for example a membrane comprising a flavorant. The packet is configured to rupture upon impact at a predetermined impact level. Advantageously, rupture of the packet releases the flavorant into the oral cavity of the subject indicating that an impact of the predetermined force has occurred. Upon release of the flavorant, the subject can withdraw from play and/or seek medical attention. In one embodiment, release of the flavorant occurs when the subject receives an impact of 90-100 Gs or occurs in response to an impact of 20 Gs, 30 Gs, 40 Gs, 50 Gs, 60 Gs, 70 Gs, 80 Gs, 90 Gs, or 100 Gs.

In particular embodiments, the flavorant is released at 60 Gs, 70 Gs, 80 Gs, or 90 Gs. In one embodiment the packet is made of a membrane where the structural integrity of the membrane is varied to burst upon impact at a desired force. The sensitivity of the membrane to impact can be varied according to the age, size, prior history, or medical condition of the subject. In one embodiment, the sensitivity of the membrane to rupture is tailored to customer specifications. Membranes susceptible to rupture are known in the art and described, for example, in U.S. Pat. No. 8,637,080. In one embodiment, the membrane comprising the flavorant is semi-permeable.

Many common materials known by those of ordinary skill in the art are suitable for use as a membrane subject to rupture upon impact. Exemplary materials include cellulose esters, cellulose ethers, cellulose esters-ethers and combinations thereof. Other materials for making the membrane include a member selected from the group consisting of cellulose acylate, cellulose diacylate, cellulose triacylate, cellulose acetate, cellulose diacetate, cellulose triacetate, mono, di and tricellulose alkanylates, mono, di and tricellulose aroylates, and the like. Exemplary polymers include cellulose acetate, cellulose trivalerate, cellulose trilaurate, cellulose tripalmitate, cellulose tri succinate, and cellulose trioclanoate; cellulose diacylates, cellulose disuccinate, cellulose dipalmitate, cellulose dioclanoate, cellulose dipentalate, and the like.

As used herein, the term “flavorant” is intended to mean a substance used to impart a pleasant flavor to the oral cavity of a subject. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, th oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. Flavors will be present in any amount as desired by those of ordinary skill in the art.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A mouth guard comprising
a U-shaped structure having an inner wall and an outer wall, wherein the inner and outer walls are connected to each other by a base forming a channel between said inner and outer walls, which are designed for the upper teeth of a user, wherein the mouth guard comprises an impact indicator.

2. The mouth guard of claim 1, wherein the outer wall of the U-shaped structure comprises one or more impact detectors.

3. The mouth guard of claim 1, wherein a shield extends from the center of the outer wall of the U-shaped structure, and the shield comprises one or more impact detectors.

4. The mouth guard of claim 3, wherein a strap extends from the center of the shield and the strap comprises one or more impact detectors.

5. The mouth guard of claim 1, wherein the U-shaped structure comprises a conduit extending between the inner and outer walls to facilitate breathing or drinking through the shield.

6. The mouth guard of claim 1, wherein the impact detector is embedded in or adheres to the outer wall or the inner wall of the U-shaped structure.

7. The mouth guard of claim 1, wherein the impact detector provides a visual signal or flavor signal indicating that an impact of a predetermined level has occurred.

8. The mouth guard of claim 1, wherein the impact detector provides a visual signal or flavor signal indicating that an impact of between 20 Gs and 105 Gs has occurred.

9. The mouth guard of claim 1, wherein the impact detector provides a visual signal at a force selected from the group consisting of 20 Gs, 50 Gs, 60 Gs, 80 Gs, 90 Gs, and 100 Gs.

10. The mouth guard of claim 1, wherein the mouth guard comprises 2 or more impact detectors each of which provides a different visual signal or flavor signal at a predetermined level of force.

11. The mouth guard of claim 1, wherein one impact detector provides a visual signal at a force under 60 Gs and the second impact detector provides a visual signal at a force greater than 60 Gs.

12. The mouth guard of claim 1, wherein the impact detector comprises a flavor packet that ruptures upon impact at a predetermined level.

13. The mouth guard of claim 1, wherein the flavor packet ruptures at a force selected from the group consisting of 20 Gs, 50 Gs, 60 Gs, 80 Gs, 90 Gs, and 100 Gs.

14. The mouth guard of claim 1, wherein the impact detector comprises liquid surface tension technology.

15. A method for detecting a force of a predetermined level applied to a subject, the method comprising contacting a subject wearing the mouth guard of claim 1 with a force, and detecting the presence or absence of a visual signal in the impact detector, thereby indicating that the force applied was at or was not at the predetermined level.

16. The method of claim 15, wherein the presence of a visual sign indicates the force applied to the subject was sufficient to cause a concussion and the absence of a visual signal indicates the force applied to the subject was not sufficient to cause a concussion.

17. A method for detecting a concussion in a subject, the method comprising contacting a subject wearing the mouth guard of claim 1 with a force, and detecting the presence or absence of a visual signal in the impact detector, wherein the presence of a color change indicates that the subject has sustained a concussion and the absence of a color change indicates that the subject has not sustained a concussion.

18. A method for a subject to detect a force of a predetermined level applied to the subject, the method comprising contacting a subject wearing the mouth guard of claim 1 with a force, and detecting the presence or absence of a flavor signal, wherein the presence of a flavor signal indicates that the force applied was at the predetermined level and the absence of a flavor signal indicates that the force applied was below the predetermined level.
